(19) United States
(12) Patent Application Publication
(10) Pub. No.: US 2004/0138503 A1
(43) Pub. Date: Jul. 15, 2004

(54) Surface modification of carbonaceous materials with TRI substituted aminoalkyl substituents

(76) Inventors: Bollepalli Srinivas, Marietta, GA (US); Jorge Ayala, Kennesaw, GA (US); Anderson O. Dotson, Marietta, GA (US)

Correspondence Address: NEEDLE & ROSENBERG, P.C., SUITE 1000, 999 PEACHTREE STREET, ATLANTA, GA, 30309-3915, US

(21) Appl. No.: 10/341,075

(22) Filed: Jan. 13, 2003

(51) Int. Cl. C07C 237/30

(52) U.S. Cl. 564/156

(57) ABSTRACT

The present invention relates to the surface modification of various carbonaceous materials, compounds and compositions. More specifically, the invention provides methods for introducing amide functionality on to the surface of carbonaceous materials, compounds and compositions, and similarly provides several surface modified carbonaceous materials resulting therefrom.

### FIELD OF THE INVENTION

The present invention relates to the surface modification of various carbonaceous materials, compounds and compositions. More specifically, the invention provides methods for introducing an amide functionality on to the surface of carbonaceous materials, compounds and compositions, and similarly provides several surface modified carbonaceous materials resulting therefrom.

### BACKGROUND OF THE INVENTION

The surface modification of carbonaceous compounds has been widely explored as a means for achieving desired chemical and physical properties not normally exhibited by carbonaceous compounds. Recently, there has been a considerable interest in surface modification of carbonaceous materials to obtain improved physiochemical properties for applications in rubber, plastics, coatings and inks.

Traditionally, various additives, dispersants and surfactants were used to improve properties of these carbonaceous materials, such as carbon black and other pigment compositions. However, the incorporation of these additional materials only provides marginal improvement in the desired properties, not to mention the added costs associated therewith. To this end, the concept of surface modification of carbonaceous materials by chemically affixing specific organic functional groups can be used to achieve and or optimize specifically desired properties.

For example, various methods for oxidizing carbon black pigment have been used to generate surface active hydroxyl and carboxylic functional sites. However, in the past, the concentration of these surface active sites has been very low, thus rendering these methods ineffective for substantially improving properties of the carbonaceous materials and for reducing the need for additional additives, dispersants, surfactants and the like. Moreover, these processes typically lead to random surface substitutions and therefore provide mixtures of carbokylic, phenolic and keto functionalities, often resulting in relatively highly acidic carbonaceous materials that are sometimes detrimental for use in the intended applications.

Therefore, as an object of the present invention, a method has been developed for chemically affixing amide functionalities onto the surface of carbonaceous materials which, in turn, provides the ability to introduce a higher and more uniform population of desired functionality, e.g., a hydroxyl substituent, onto the surface of the carbonaceous material in a more uniform and controlled manner. More importantly, the methods developed and discussed herein, provide for the substitution of carboxylic functionalities with less acidic functional groups that can advantageously provide an increased potential for interaction with substrates and therefore result in improved properties for use in rubber, plastics, coatings and ink applications.

### SUMMARY OF THE INVENTION

Among other aspects, the present invention is based upon methods for introducing amide functionalities onto the surface of carbonaceous materials, compounds and compositions and similarly provides several surface modified carbonaceous materials resulting therefrom.

In a first aspect, the present invention provides a surface modified carbonaceous material comprising a carbonaceous material having a plurality of amide functionalities of the general formula —(CO)—NH—R—CR¹R²R³, surface bonded thereto, wherein R is a single bond or a straight chain C₁-C₁₂ alkyl, and wherein R¹, R², and R³ are independently selected from C₁-C₁₂ hydroxy alkyl, C₁-C₁₂ hydroxy alkenyl and C₁-C₁₂ hydroxy alkynyl, hydrogen, hydroxyl, and C₁-C₁₂ alkyl.

In a second aspect, the present invention provides a process for the manufacture of a surface modified carbonaceous material comprising a carbonaceous material having plurality of amide functionalities of the general formula —(CO)—NH—R—CR¹R²R³, surface bonded thereto, comprising the steps of providing a carbonaceous material comprising a plurality of carboxylic acid functional groups surface bonded thereto; and reacting the carbonaceous material with an amine of the general formula H₂N—R—CR¹R²R³, wherein R is a single bond or straight chain C₁-C₁₂ alkyl, and wherein R¹, R², and R³ are independently selected from C₁-C₁₂ hydroxy alkyl, C₁-C₁₂ hydroxy alkenyl and C₁-C₁₂ hydroxy alkynyl, hydrogen, hydroxyl, and C₁-C₁₂ alkyl. The reaction of the carbonaceous material with the amine proceeds under conditions effective to provide a surface modified carbonaceous material comprising a plurality of amides of the general formula —(CO)—NH—R—CR¹R²R³, surface bonded thereto, wherein R is a single bond or straight chain C₁-C₁₂ alkyl, and wherein R¹, R², and R³ are independently selected from C₁-C₁₂ hydroxy alkyl, C₁-C₁₂ hydroxy alkenyl and C₁-C₁₂ hydroxy alkynyl, hydrogen, hydroxyl, and C₁-C₁₂ alkyl.

In a third aspect, the present invention provides several end use applications and formulations comprising the surface modified carbonaceous materials of the present invention. For example, in one embodiment, the present invention provides an aqueous composition comprising the surface modified carbonaceous materials of the present invention and water. In a second embodiment, the present invention provides an elastomeric composition comprising the surface modified carbonaceous compounds of the present invention and an elastomer.

Additional advantages of the invention will be obvious from the description, or may be learned by practice of the invention. Additional advantages of the invention will also be realized and attained by means of the elements and combinations particularly pointed out in the appended claims. Therefore, it is to be understood that both the foregoing general description and the following detailed description are exemplary and explanatory of certain embodiments of the invention, and are not restrictive of the invention as claimed.

### BRIEF DESCRIPTION OF THE FIGURES

The appended Figures, which are incorporated in and constitute part of the specification, illustrate the effectiveness of the process of the present invention to provide a surface modified carbonaceous compound having a plurality of amide functionalities surface bonded thereto.

FIG. 1 is a plot of the XPS spectrum of the oxidized carbon black used to prepare the surface modified carbonaceous product of Example 1.

FIG. 2 is a plot of the XPS spectrum of the surface modified carbon black produced in Example 1.

FIG. 3 is a plot of the XPS spectrum indicating the nature of the surface bonded oxygen groups identified in FIG. 1.

FIG. 4 is a plot of the XPS spectrum indicating the nature of the surface bonded oxygen groups identified in FIG. 2.

FIG. 5 is a plot of the aggregate size and aggregate size distribution of the TRIS modified Raven 5000 Ultra II dispersed in the waterborne acrylic composition of Example 7(b).

FIG. 6 is a plot of the aggregate size and aggregate size distribution of an unmodified Raven 5000 Ultra II dispersed in the waterborne acrylic composition of Example 7(a).

### DETAILED DESCRIPTION OF THE INVENTION

The present invention may be understood more readily by reference to the following detailed description and any examples provided herein. It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular forms “a,” “an,” and “the” comprise plural referents unless the context clearly dictates otherwise. For example, reference to a component in the singular is intended to comprise a plurality of components.

Ranges may be expressed herein as from “about” or “approximately” one particular value and/or to “about” or “approximately” another particular value. When such a range is expressed, another embodiment comprises from the one particular value and/or to the other particular value. Similarly, when values are expressed as approximations, by use of the antecedent “about,” it will be understood that the particular value forms another embodiment.

As used herein, a weight percent of a component, unless specifically stated to the contrary, is based on the total weight of the formulation or composition in which the component is included.

As used herein, the term “alkyl” refers to a paraffinic hydrocarbon group, which may be derived from an alkane by dropping one hydrogen from the formula. Non-limiting examples include C₁-C₂₀ alkane derivatives such as methyl, ethyl, propyl, isopropyl, butyl, t-butyl, and isobutyl. To this end, it should be understood that an alkyl substituent suitable for use in the present invention can be a branched or straight chain alkyl substituent.

As used herein, the term “alkenyl” is intended to refer to a substituent derived from the class of unsaturated hydrocarbons having one or more double bonds. Those containing only one double bond are referred to as alkenes or alkenyl substituents. Those with two or more double bonds are called alkadienes (alkadienyl), alkatrienes (alkatrienyl) and so on. Non-limiting examples include ethylene, propylene, butylene and the like. To this end, it should be understood that an alkenyl substituent suitable for use in the present invention can be substituted or unsubstituted.

As used herein, the term “alkynyl” is intended to refer a substituent derived from the class of unsaturated hydrocarbons having one or more triple bonds.

As used herein, the term “surface bonded” refers to a substituent that is substantially bonded, either covalently or ionically, only to the outer surface of the carbonaceous compound particle. To this end, a substituent that is “surface bonded” is substantially absent from the inner region or core of the carbonaceous compound particle.

As used herein, the term “optional” or “optionally” means that the subsequently described event or circumstance may or may not occur, and that the description includes instances where said event or circumstance occurs and instances where it does not. For example, the phrase “optionally substituted lower alkyl” means that the lower alkyl group may or may not be substituted and that the description includes both unsubstituted lower alkyl and lower alkyl where there is substitution.

As used herein, by use of the term “effective,” “effective amount,” or “conditions effective to” it is meant that such amount or reaction condition is capable of performing the function of the compound or property for which an effective amount is expressed. As will be pointed out below, the exact amount required will vary from one embodiment to another, depending on recognized variables such as the compounds employed and the processing conditions observed. Thus, it is not always possible to specify an exact “effective amount” or “condition effective to.” However, it should be understood that an appropriate effective amount will be readily determined by one of ordinary skill in the art using only routine experimentation.

As used herein, the term “XPS” refers to X-ray Photoelectron Spectroscopy. Accordingly, all XPS measurements disclosed herein have been conducted using the Physical Electronics 5802 Multitechnique with Al Ka X-ray source.

As used herein, the term “carbonaceous material” is intended to include, without limitation, i) carbonaceous compounds having a single definable structure; or ii) aggregates of carbonaceous particles, wherein the aggregate does not necessarily have a unitary, repeating, and/or definable structure or degree of aggregation. For example, a carbon black material as used herein can be a carbon black compound having a definable structure or, alternatively, can also be an aggregate of carbonaceous particles wherein the exact structure or degree of aggregation is unknown.

As initially set forth above, the present invention relates to methods for introducing amide functionalities onto the surface of various carbonaceous materials. To that end, in a first aspect, the present invention provides a process for the manufacture of a surface modified carbonaceous material comprising a plurality of amides of the general formula, —(CO)—NH—R—CR¹R²R³, surface bonded thereto, wherein R is a single bond or straight chain C₁-C₁₂ alkyl, and wherein R¹, R², and R³ are independently selected from C₁-C₁₂ hydroxy alkyl, C₁-C₁₂ hydroxy alkenyl, C₁-C₁₂ hydroxy alkynyl, hydrogen, hydroxyl, and C₁-C₁₂ alkyl.

Accordingly, the process comprises the steps of providing a carbonaceous material comprising a plurality of carboxylic acid functional groups surface bonded thereto; and then reacting the carbonaceous material with an amine of the general formula H₂N—R—CR¹R²R³, wherein R is a single bond or straight chain C₁-C₁₂ alkyl and wherein R¹, R², and R³ are independently selected from C₁-C₁₂ hydroxy alkyl, C₁-C₁₂ hydroxy alkenyl, C₁-C₁₂ hydroxy alkynyl, hydrogen, hydroxyl, and C₁-C₁₂ alkyl; wherein the reaction of the carbonaceous material with the amine proceeds under conditions effective to provide a surface modified carbonaceous material comprising a carbonaceous material having a plurality of amides of the general (formula —(CO)—NH—R—CR¹R²R³, surface bonded thereto, wherein R is a single bond or straight chain C₁-C₁₂ alkyl, and wherein R¹, R², and R³ are independently selected from C₁-C₁₂ hydroxy alkyl, C₁-C₁₂ hydroxy alkenyl, C₁-C₁₂ hydroxy alkynyl, hydrogen, hydroxyl, and C₁-C₁₂ alkyl.

In accordance with this and other aspects to be discussed below, the process of the present invention can be used with a variety of carbonaceous compounds and/or materials. To this end, any carbonaceous compound or material can be used provided there are sufficient reactive carboxylic acid functional sites capable of interacting with a suitable amine under conditions effective to provide a desired surface modified carbonaceous material. For example, in one aspect, the carbonaceous material is an oxidized carbonaceous carbon black composition, including several products available from Columbian Chemicals Company, Marietta, Ga., 30062 U.S.A., such as the RAVEN 7000, 5750, 5250, 5000 (Ultra II and Ultra III), 3500, 1255, 1100 Ultra, 1080 Ultra, 1060 Ultra, 1040, and 1035.

In an alternative aspect, the process of the present invention can also comprise the use of non-oxidized carbonaceous materials that ordinarily lack sufficient reactive carboxylic acid functional sites, such as the N121, N234 and N339 ASTM tread grade carbon blacks, also available from Columbian Chemicals Company, Marietta, Ga., 30062 U.S.A. To this end, it will be appreciated by one of ordinary skill in the art that if it is desired to conduct the process of the present invention on an initially non-oxidized carbonaceous material, the process will further comprise a step of pre-oxidizing the carbonaceous material to thereby provide a carbonaceous material having sufficient reactive carboxylic acid functional sites capable of interacting with a suitable amine under conditions effective to provide a desired surface modified carbonaceous material. Examples of suitable pre-oxidizing reaction processes can be found in U.S. Pat. Nos. 3,959,008, 4,075,140, 6,120,594 and 6,471,933, the disclosures of which are incorporated herein by this reference in their entireties for all purposes.

Although not required, it is preferred that the carbonaceous compound have a surface area of at least approximately 25 m²/g as measured by ASTM-D4820. In a more preferred aspect, when measured by ASTM-D4820, the carbonaceous compound will have a surface area of at least approximately 100 m²/g. In still a more preferred aspect, the surface area of the carbonaceous compound will be greater than approximately 200 m²/g when measured according to the ASTM-D4820 method.

Specific examples of suitable carbonaceous compounds include, without limitation, carbon fiber, activated charcoal, finely divided carbon, carbon black, graphite, fullerenic carbons, and nanocarbons. In a preferred aspect, the carbonaceous material is a carbon black having a surface area greater than approximately 200 m²/g and an oil adsorption rate of at least 60 ml/100 g as measured by ASTM-D2414.

As indicated above, suitable amines for use with the process of the present invention have the generic formula H₂N—R—CR¹R²R³. To this end, R represents either a single bond or straight chain C₁-C₁₂ alkyl moiety. Similarly, R¹, R², and R³ are each independently selected from C₁-C₁₂ hydroxy alkyl, C₁-C₁₂ hydroxy alkenyl, C₁-C₁₂ hydroxy alkynyl, hydrogen, hydroxyl, and C₁-C₁₂ alkyl moieties. It is understood that the particular amine selected will ultimately be dependent on the particular functionality and corresponding chemical and physical properties desired.

In a preferred aspect, at least one of R¹, R², and R³ comprises a hydroxyl functionality. In still a more preferred aspect, at least two of R¹, R², and R³ comprise a hydroxyl functionality. To this end, in still a more preferred aspect, the amine is tris(hydroxymethyl)aminomethane (TRIS), which advantageously is capable of substituting a carboxylic acid functionality with an amide functionality comprising three hydroxyl functionalities. The TRIS is commercially available from Aldrich Chemical Company.

When tris(hydroxymethyl)aminomethane or other hydroxy containing amine is used with the present process, the surface atomic concentration of oxygen surface bonded to the carbonaceous materials advantageously increases by at least approximately 20.0% relative to the surface atomic concentration of oxygen surface bonded to the initial oxidized carbonaceous material. In a more preferred aspect, the surface atomic concentration of oxygen surface bonded thereto increases in the range of from at least 20% to approximately 100% relative to the initial surface atomic concentration of oxygen surface bonded thereto, including such relative increases as at least approximately 25%, 30%, 35%, 40%, 45%, 50%, 55%, 60%, 65%, 70%, 75%, 80%, 85%, 90% and 95%. Moreover, the reaction is specifically targeted to surface bonded carboxylic acid functionalities and can therefore more uniformly populate an oxidized carbonaceous material with the desired functionality, e.g., a hydroxyl functionality.

In one aspect of the process, the reaction of the amine with the carbonaceous material can take place in the presence of a suitable solvent. The process according to this aspect comprises the steps of providing a carbonaceous material comprising a plurality of carboxylic acid functional groups surface bonded thereto; introducing the carbonaceous material and an amine of the general formula H₂N—R—CR¹R²R³ into a suitable solvent; and then reacting the carbonaceous material with the amine under conditions effective to provide a surface modified carbonaceous material comprising a carbonaceous material having a plurality of amide functionalities of the general formula —(CO)—NH—R—CR¹R²R³, surface bonded thereto.

According to this aspect, suitable solvents for carrying out the reaction include any non-aqueous solvent that will not interfere or compete in the reaction of the present invention. In one aspect, the preferred solvent can be an aromatic solvent such as toluene, xylene or a mixture thereof. In an alternative aspect, the preferred solvent can be an aliphatic solvent, such as the C₁ through C₉ lower alkanols, or mixtures thereof. In still another aspect, the solvent can include dimethylsulfoxide (DMSO), dimethylethanolamine (DMEA), acetonitrile, triethanolamine (TEA) or any mixture thereof. Moreover, it should be understood that any one of the above-mentioned solvents is suitable for use in the process of the present invention either alone or in combination with any one or more other solvent(s) set forth above.

It is to be understood that the preferred solvent or mixture thereof, will ultimately be dependent on the particular amine used in the reaction process and will be readily determined by one of ordinary skill in the art through no more than mere routine experimentation.

It will also be appreciated that the optimum reaction conditions for performing the process of the present invention will vary depending on the particular amine, solvent, and/or carbonaceous material selected to be surface modified. To this end, arriving at such optimum conditions will again be readily obtainable by one of ordinary skill in the art through no more than routine experimentation.

The process, as set forth above, can be successfully performed on virtually any scale, provided the reaction conditions remain effective for performing the desired surface modification reaction. To that end, in accordance with a preferred aspect, the carbonaceous material is first introduced into a desired solvent or solvent mixture such that the weight ratio of carbonaceous material relative to solvent and/or solvent mixture is in the range of from approximately 1:2 to approximately 1:5, including such preferred weight ratios as 1:2.5, 1:3, 1:3.5, 1:4 and 1:4.5.

Likewise, the desired amine is similarly dissolved in a solvent and/or solvent mixture such that the weight ratio of amine to solvent is in the range of from approximately 1:5 to approximately 1:20, including such weight ratios 1:6, 1:7, 1:8, 1:9, 1:10, 1:11, 1:12, 1:13, 1:14, 1:15, 1:16, 1:17, 1:18 and 1:19. It should be understood that certain amines suitable for use in the reaction process are only sparingly soluble in a selected solvent and/or solvent mixture. Therefore, under such circumstances the presence of excess solvent can advantageously increase the dissolution of the amine in the desired solvent and/or solvent mixture.

The resulting mixture of the at least substantially dissolved amine, as described above, is then added to the mixture of solvent and carbonaceous material described above and refluxed in a suitable reflux assembly for a period of time sufficient to effect a complete or at least substantially complete surface modification reaction. To this end, the optimum reaction temperature will also vary depending on the selected solvent or solvent mixture. Examples of suitable reaction temperatures include temperatures in the range of from approximately 80° C. to approximately 120° C., including such temperatures as 85° C., 90° C., 95° C., 100° C., 105° C., 110° C., and 115° C. Likewise, the duration of the reaction time can vary from as short as 1 hour up to and exceeding approximately 24 hours, which will also be dependent on the solvent or solvent mixture, amine and carbonaceous material selected. In various aspects, the reaction mixture can therefore be refluxed at a suitable temperature for periods of time, including but not limited to, from a lower limit of approximately 1 hour to an upper limit of approximately 2, 4, 6, 8, 10, 12, 14, 16, 18, 20 or 22 hours.

In an alternative aspect, the surface modification reaction of an amine with a carbonaceous material can proceed in the absence or at least substantial absence of a suitable solvent. To that end, it is not required by the invention that both the amine and the carbonaceous material be in the presence of a suitable solvent in order for the surface modification reaction to proceed successfully. Rather, the process according to this aspect provides the ability to first dissolve an amine into a suitable solvent and then simply wet down or coat the carbonaceous material with the resulting mixture of solvent and at least substantially dissolved amine. The resulting carbonaceous material that has been wet down or otherwise coated with the solvent and at least substantially dissolved amine can then be heated to a temperature sufficient to evaporate or otherwise remove the solvent or solvent mixture, melt the remaining amine, and subsequently initiate the surface modification reaction within the melted amine.

In accordance with this aspect, suitable solvents for dissolving the amine can include any aqueous or non-aqueous solvent that is capable of dissolving the desired amine and that will not interfere or compete in the reaction of the present invention. In one aspect, the preferred solvent is water. Suitable non-aqueous solvents include aromatic solvents such as toluene, xylene or a mixture thereof. Other suitable non-aqueous solvents include aliphatic solvents, such as the C₁-C₈ lower alkanols, or mixtures thereof. In still another aspect, the suitable solvent or solvent mixture can include dimethylsulfoxide (DMSO), dimethylethanolamine (DMEA), acetonitrile, triethanolamine (TEA) or any mixture thereof. It should be understood that any one of the above-mentioned solvents is suitable for use in the process of the present invention either alone or in combination with any one or more other solvent(s) set forth above.

It will be appreciated that the optimum reaction conditions for performing the above-mentioned reaction, in the absence or at least substantial absence of a solvent, will vary depending on the particular amine, solvent and the carbonaceous material selected to be surface modified. To this end, arriving at such optimum conditions will again be readily obtainable by one of ordinary skill in the art through no more than routine experimentation.

Likewise, the optimum solvent or mixture thereof will ultimately be dependent on the particular amine used in the reaction process and will be readily determined by one of ordinary skill in the art through no more than mere routine experimentation. Moreover, the optimum amount of solvent will be dependent on the amount of amine to be dissolved and, as such, said optimum amount will be known by one of ordinary skill in the art or otherwise readily obtainable through no more than routine experimentation.

The surface modification reaction of an amine with a carbonaceous material in the absence of a suitable solvent can also be successfully performed on virtually any scale, provided the reaction conditions remain effective for performing the desired surface modification reaction. In one aspect, the desired amine is first dissolved in a suitable solvent such that the amine is present in sufficient amount to react with the plurality of carboxylic hydroxyl functionalities present on an oxidized carbonaceous material. For example, a suitable amount of amine to be dissolved in the solvent is an amount that is at least approximately 10 percent by weight, relative to the amount of carbonaceous material to be reacted. However, it should be understood that any amount of amine is acceptable and, as such, the amount of amine to be dissolved in the solvent can be any amount within the range of at least approximately 5 weight percent to approximately 30 weight percent, relative to the amount of carbonaceous material to be surface modified, including such amounts as up to 10, 15, 20, or 25 weight percent.

According to this aspect, the resulting mixture of at least substantially dissolved amine and solvent is then used to wet down or coat the desired carbonaceous material. Any means known to one of ordinary skill in the art for wetting down or coating a carbonaceous material can be used to at least substantially coat the surface of the carbonaceous material with the solvent and amine mixture, including without limitation, such processes as spraying the mixture onto the carbonaceous material.

Once the carbonaceous material has been at least substantially wet down or coated, the carbonaceous material is then heated to a temperature equal to or exceeding the melting point of the previously dissolved amine, which first boils off or otherwise removes the solvent and further initiates the surface modification reaction to proceed in the presence of the melted amine. To this end, the optimum reaction temperature will vary depending on the selected amine. Examples of suitable reaction temperatures for this aspect include temperatures in the range of from approximately 170° C. to approximately 200° C., including such temperatures as 175° C., 180° C., 185° C., 190° C., and 195° C.

In accordance with this aspect, it should be understood that the duration of the reaction time will again be dependent on the particular solvent or solvent mixture, amine and carbonaceous material selected. To that end, the optimum reaction time will be the same or substantially similar to the reaction times in those embodiments previously set forth above and will be readily obtainable by one of ordinary skill in the art through no more than routine experimentation.

Once the surface modification reaction is at least substantially complete, the resulting surface modified carbonaceous material comprising a plurality of amide functionalities can optionally be washed one or more times with ethanol and then subsequently with water, if desired. Following the wash, the surface modified product can further be filtered and then dried at a temperature of at least approximately 110° C. for a period of time effective to obtain at least substantially dried, purified surface modified carbonaceous product.

The degree of success of the reaction can be measured by recording the XPS spectra of the finished product. To this end, the surface modified carbonaceous material will exhibit a peak representative of nitrogen species that are surface bonded to the carbonaceous material as a result of the amide formation reaction. As such, in a preferred aspect, greater than approximately 50% of the initial plurality of carboxylic acid functional groups have been reacted to provide the plurality of amide groups. In still a more preferred aspect, greater than at least 70% of the initial plurality of carboxylic acid functional groups have been reacted to provide the plurality of amide groups. And, in still a more preferred aspect, greater than at least 90% of the initial plurality of carboxylic acid functional groups have been reacted to provide the plurality of amide groups.

Having set forth process components of the present invention, it follows that in an alternative aspect, the present invention also provides for several surface modified carbonaceous materials resulting from the aforementioned process.

Therefore, in a second aspect, the present invention further provides a surface modified carbonaceous material, comprising a carbonaceous material having a plurality of amide functionalities of the general formula —(CO)—NH—R—CR¹R²R³, surface bonded thereto, wherein R is a single bond or a straight chain C₁-C₁₂ alkyl and wherein R¹, R², and R³ are independently selected from C₁-C₁₂ hydroxy alkyl, C₁-C₁₂ hydroxy alkenyl, C₁-C₁₂ hydroxy alkynyl, hydrogen, hydroxyl, and C₁-C₁₂ alkyl.

As previously discussed in connection with the process aspects described above, the carbonaceous compound or material is preferably any carbonaceous material that has a surface area of at least approximately 25 m²/g as measured by ASTM-D4820. In a more preferred aspect, when measured by ASTM-D4820, the carbonaceous material has a surface area of at least approximately 100 m²/g. In still a more preferred aspect, the surface area of the carbonaceous material is greater than approximately 200 m²/g when measured according to the ASTM-D4820 method.

To this end, examples of suitable carbonaceous materials include, without limitation, carbon fiber, activated charcoal, finely divided carbon, carbon black, graphite, fullerenic carbons, and nanocarbons. Moreover, in a preferred aspect, the carbonaceous material is an oxidized carbon black having a surface area greater than approximately 200 m²/g and an oil adsorption rate of at least 60 ml/100 g as measured by ASTM-D2414.

As stated above, R is selected from a single bond or a straight chain C₁-C₁₂ alkyl. Likewise, functional groups R¹, R², and R³ are each independently selected from C₁-C₁₂ hydroxy alkyl, C₁-C₁₂ hydroxy alkenyl, C₁-C₁₂ hydroxy alkynyl, hydrogen, hydroxyl, and C₁-C₁₂ alkyl. However, in a preferred aspect, R is a single bond and at least one of R¹, R², and R³ comprises a hydroxyl susbtituent. In still a more preferred aspect, R is again selected to be a single bond, and at least two of functional groups R¹, R², and R³ comprise a hydroxyl substituent. To this end, in a most preferred aspect, the surface modified carbonaceous material comprises a plurality if amide functionalities of the generic formula —(CO)—NH—R—CR¹R²R³, wherein R is a single bond and each of R¹, R², and R³ individually represent a hydroxy methyl substituent.

It will be appreciated upon practicing the present invention that the surface modified carbonaceous materials resulting therefrom exhibit several advantageously improved characteristics over the initial, unmodified, carbonaceous material. For example, in the preferred aspect discussed above, wherein the surface modified carbonaceous material comprises a plurality of amide functionalities of the generic formula (CO)—NH—R—CR¹R²R³, and wherein R is a single bond and each of R¹, R², and R³ individually represent a hydroxy methyl substituent, the process effectively uniformly populates one equivalent of a carboxylic functionality with three equivalents of hydroxyl functionalities.

To that end, the population of surface bonded oxygen resulting from these added hydroxyl functionalities can be directly measured through X-ray Photoelectron Spectroscopy (XPS). Preferably, the surface modified carbonaceous materials of the present invention will have a surface atomic concentration of oxygen, as measured by XPS, of at least approximately 8.0% relative to the total surface atomic concentration of the surface modified carbonaceous material.

Likewise, the presence of the surface bonded amide functionality can also be indicated by an XPS measurement of surface bonded nitrogen. Accordingly, the surface modified carbonaceous materials of the present invention preferably have a surface atomic concentration of nitrogen that is greater than at least approximately 0.5%. In a more preferred aspect, the surface atomic concentration of nitrogen will be in the range of from at least approximately 0.1% to at least 0.6% relative to the total surface atomic concentration of the surface modified carbonaceous material, including such concentrations as at least approximately 0.2%, 0.3%, 0.4% and 0.5%. In still a more preferred aspect, the surface atomic concentration of nitrogen is greater that at least approximately 0.9% relative to the total surface atomic concentration of the surface modified carbonaceous composition.

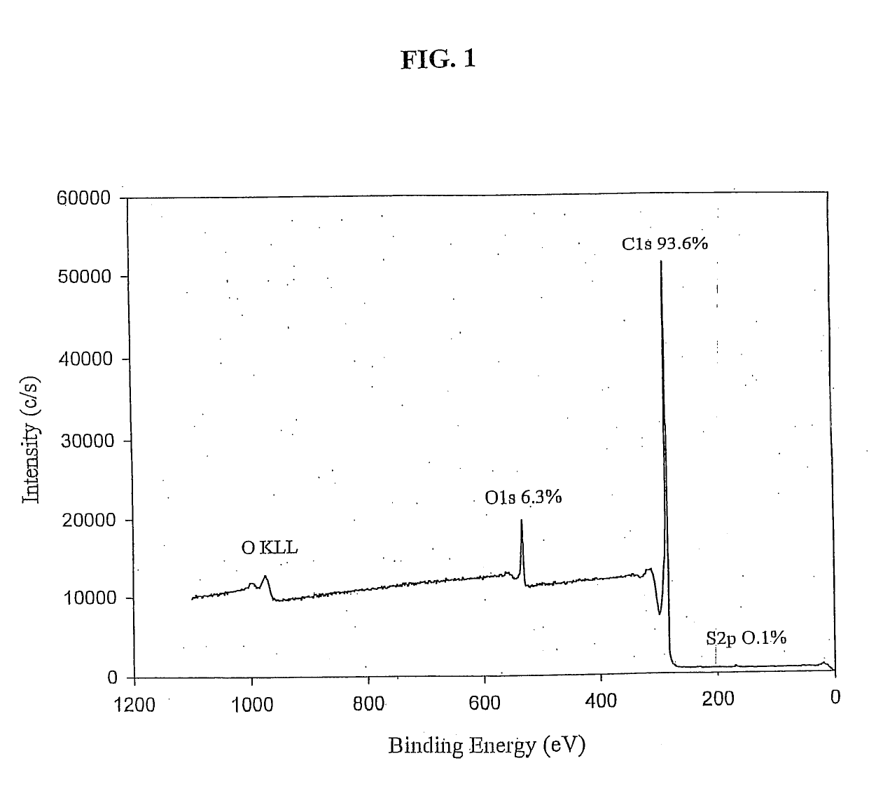

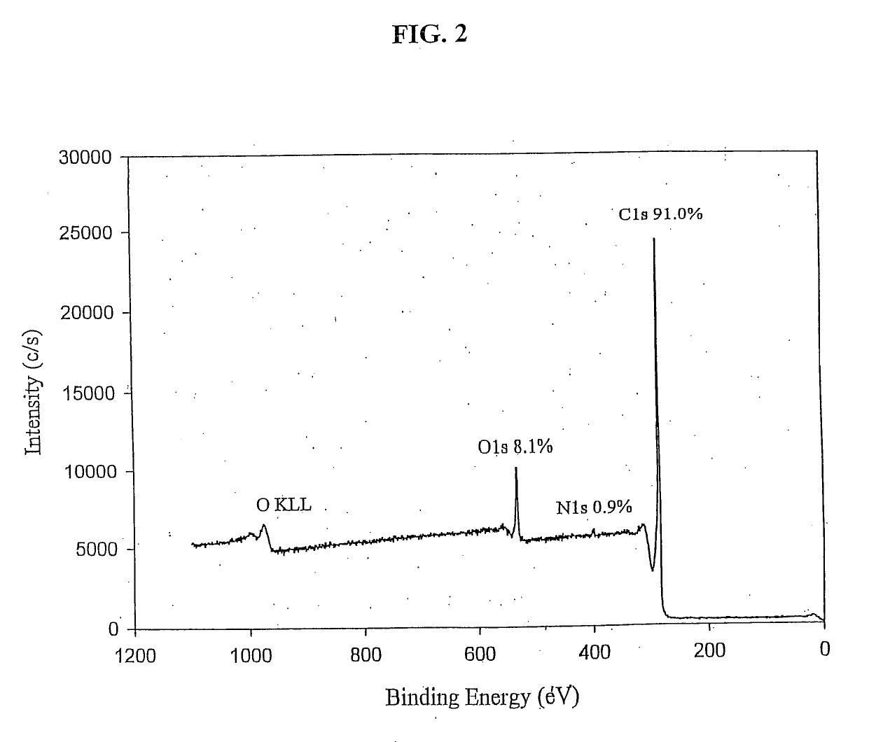

For example, with specific reference to the appended Figures, FIGS. 1 and 2 each plot the XPS spectra of the oxidized carbon black used in Example 1 and the resulting TRIS modified carbon black prepared by Example 1, respectively. The spectra indicate the surface atomic concentrations of carbon, oxygen and nitrogen surface bonded to the carbon black composition. As can be seen in FIG. 2, the TRIS modified carbon black composition prepared in Example 1 has a surface atomic concentration of oxygen of approximately 8.1%. This compares to the unmodified oxidized carbon black composition depicted in FIG. 1, having a surface atomic concentration of oxygen of approximately 6.3%.

Likewise, as can be seen again in FIG. 2, the TRIS modified carbon black composition prepared in Example 1 has a surface atomic concentration of nitrogen of approximately 0.9%. This compares to the unmodified oxidized carbon black composition depicted in FIG. 1, having a less than measurable surface atomic concentration of nitrogen species.

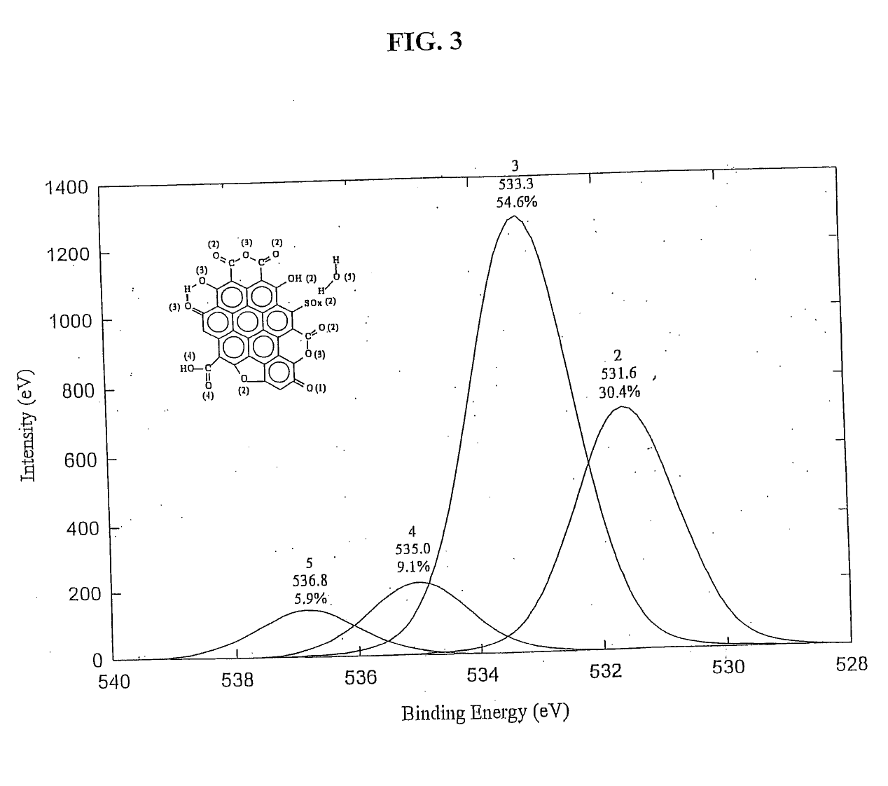

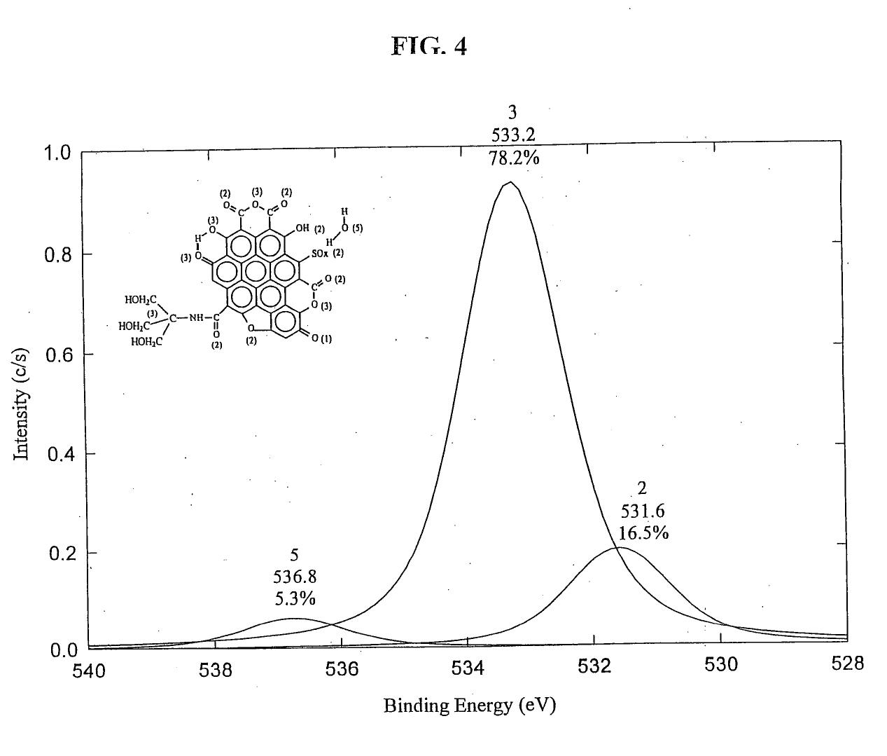

Turning to FIGS. 3 and 4, XPS spectra can also be used to verify that the relative increase in surface atomic concentration of oxygen, as indicated by comparison of FIGS. 1 and 2, is a result of the substitution of the carboxylic hydroxyl functionality with a tris(hydroxymethyl)amino methane functionality.

More specifically, FIG. 3 represents the XPS spectrum of oxygen region of the oxidized carbon black used in Example 1. The plot indicates four peaks corresponding to four measurable oxygen species surface bonded to the unmodified carbon black of Example 1. As can be seen, peak (4), having a binding energy of approximately 535 (eV), represents oxygen present within the surface bonded carboxylic oxygen substituents and measures approximately 9.1% of the total oxygen species surface bonded to the unmodified carbon black. Likewise, peak (3), having a binding energy of approximately 533.3 (eV), represents additional surface bonded anhydride and ketonic oxygen species, generally unavailable for surface modification reactions, and measures approximately 54.6% of the total oxygen species surface bonded to the unmodified carbon black.

With reference to FIG. 4, which represents the XPS spectrum of the oxygen region of the TRIS modified carbon black prepared in Example 1, when viewed in comparison to FIG. 3, described above, the XPS data indicates that the surface bonded carboxylic oxygen substituents present on the unmodified carbon black and represented by peak (4) on FIG. 3, has been depleted and thus indicates that said carboxylic oxygen functionality was the target of the tris(hydroxymethyl)amino methane substitution reaction. Moreover, peak (3) has increased to 78.2% from 54.6%, also attributed to the presence of the hydroxyloxygen species present within the tris(hydroxymethyl)amino methane group, which have a binding energy approximately equal to that of the previously measured surface bonded anhydride and ketonic oxygen species.

Thus, it will be appreciated that the ability to uniformly populate the carbonaceous material with a desired functionality, such as a hydroxyl functionalities, can provide carbonaceous materials having substantially improved properties, such as facilitating chemical interactions with coupling agents and enhancing the relative ease of further chemical reactions and processing. Moreover, the relative polarity of the carbonaceous material can likewise be uniformly adjusted to obtain compatibility with specifically desired solvents and mediums. To this end, the surface modified carbonaceous materials of the present invention can provide excellent compatibility with highly polar solvents such as ketones, esters and the like. Additionally, the surface modified carbonaceous materials exhibit improved wetability and rheological properties making them similarly well suited for use in aqueous dispersions and other waterborne systems.

In view of these advantageous properties, in still another aspect, the present invention further provides several end use formulations and applications for the surface modified carbonaceous compounds set forth above.

To this end, the modified carbonaceous materials of the present invention are useful in virtually any formulation wherein a carbonaceous material, such as carbon black, is used. These surface modified carbonaceous materials are expected to be particularly useful in applications where the substantially uniform introduction and/or increased population of hydroxyl functionalities are expected to produce improved physical and/or chemical properties of the carbonaceous materials within the particular end use formulations, and in turn providing an end use product having improved performance properties attributed thereto. For example, the surface modified carbonaceous materials of the present invention are suitable for use in plastics, elastomers, polyurethane coatings, acrylic coatings, inks, automotive coatings and automotive polymeric and/or plastic systems.

One such application of the surface modified carbonaceous materials of the present invention is its use as a filler material in an elastomeric polymer composition, such as a rubber composition for use in tires. According to this aspect, the modified carbonaceous materials of the present invention, when used in elastomeric systems, are more capable of reacting with silane coupling agents and therefore are particularly well suited for use as a filler material in various rubber applications where it is desired to have the filler coupled to a coupling agent and thereby to the polymeric compositions.

It is known that silane coupling agents, when used in conjunction with silica filler, can promote significant improvements in rubber compositions. For example, silica is a viable filler for rubber compositions that can advantageously provide for the decreased beat buildup of a rubber composition under test conditions. Additionally, coupling the silica filler to the elastomeric polymer results in reductions in dynamic tangent delta at 60° C., which in turn correlates to a lowered rolling resistance in tire applications. These improvements and others come from enhancements in the dispersability of the silica filler as well as from coupling of the elastomeric polymer to the surface of the silica through the use of silane coupling agents.

While carbon black has been used as a filler material in elastomeric compositions, carbon black compositions having a high surface area and/or a low structure can be very difficult to disperse. As a result, improvements in carbon black dispersion through the use of coupling agents, which function similar to the manner in which silica coupling agents function, would be desirable. To that end, the surface modified carbonaceous materials of the present invention are more capable of reacting with silane coupling agents and therefore can provide improved dispersibility when used as a filler material.

The following reaction schemes illustrate one example of how the surface modified carbonaceous materials of the present invention could be used as a filler material in combination with a silane coupling agent in an elastomeric formulation. Scheme 1 illustrates how a silica filler “10” reacts with a Silane coupling agent “20” to form an intermediate “30” and subsequently form a “silica coupled to polymer” elastomeric composition “40”.

Likewise, Scheme 2 illustrates how a tris(hydroxymethyl)amino methane surface treated carbon black composition according to the present invention “50” reacts with a silane coupling agent “20” to form an intermediate “60” and subsequently form a “carbon black coupled to elastomeric polymer” composition “70”. As illustrated, the surface modified carbonaceous material “50” is capable of effectively replacing the use of the silica filler “10” shown in reaction scheme 1.

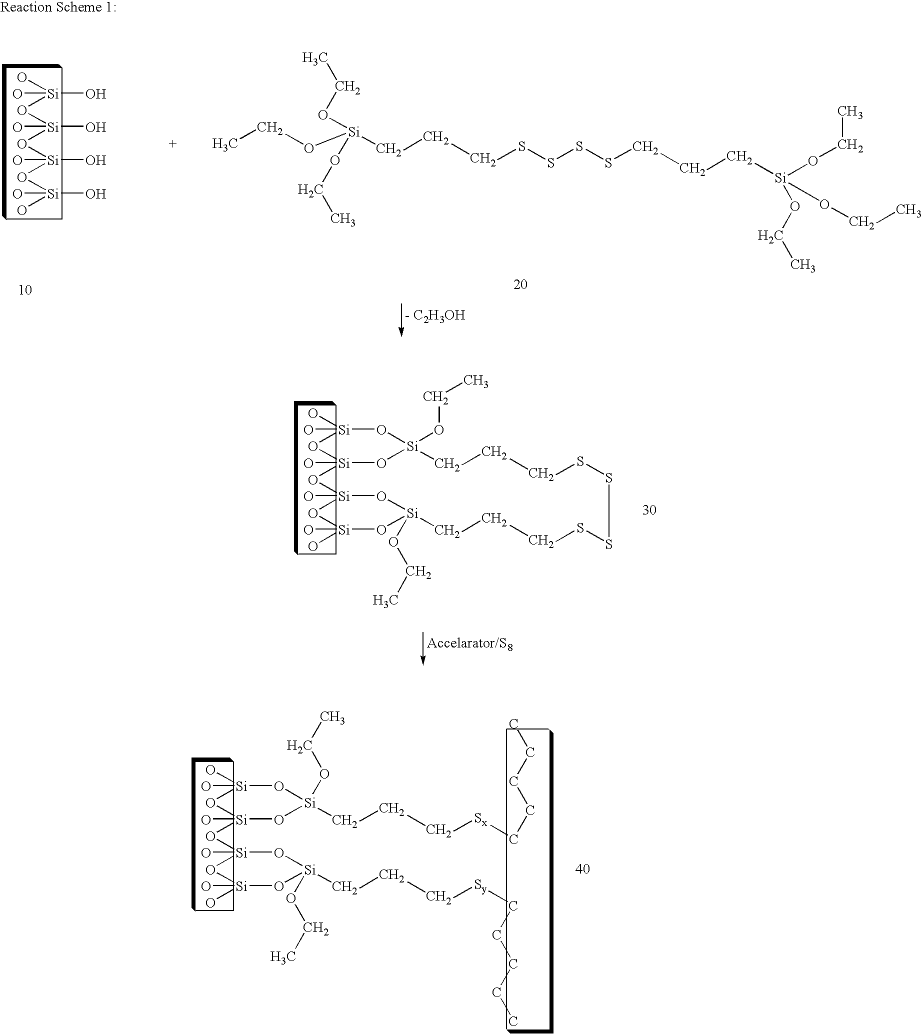

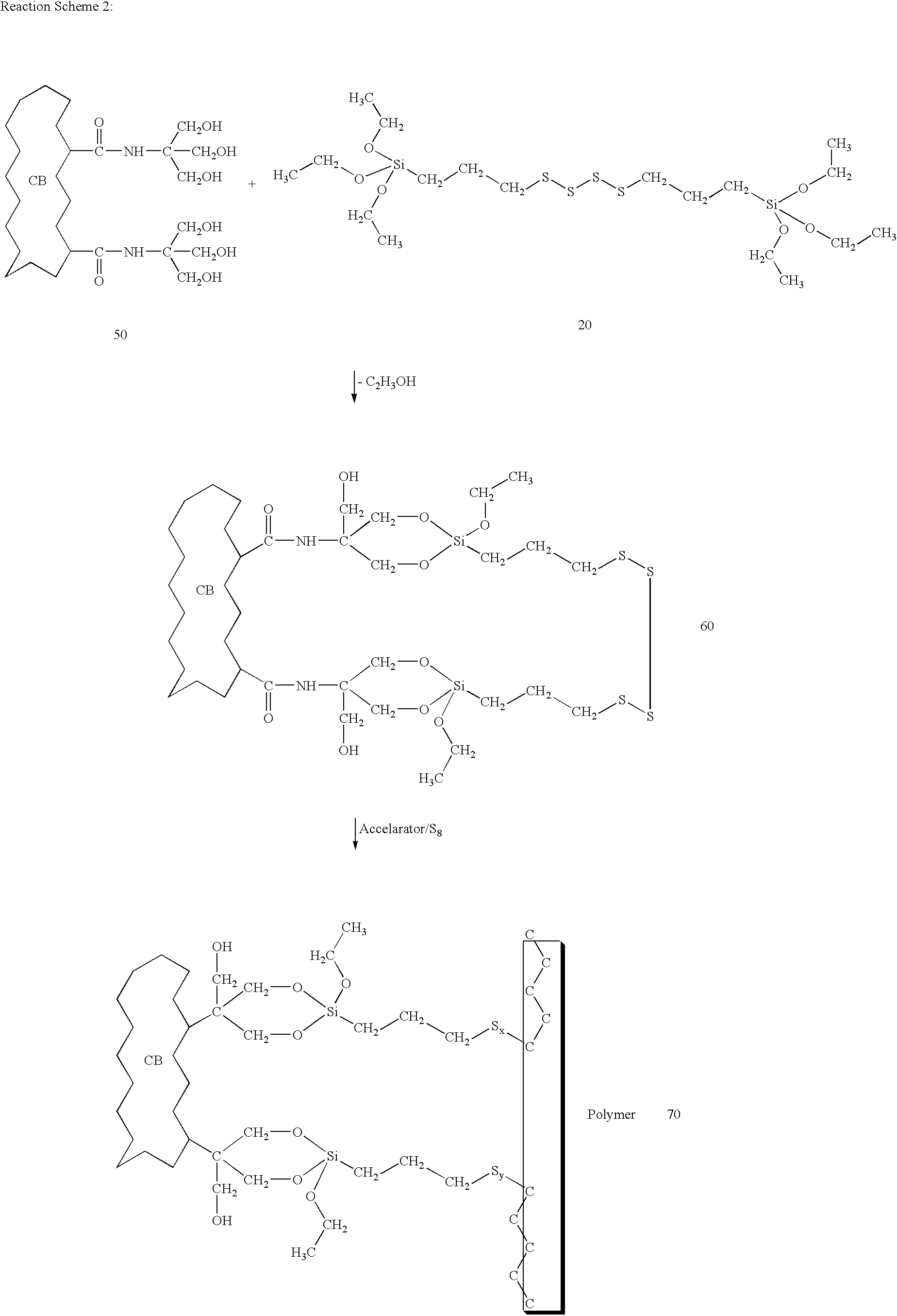

The ability to couple the elastomeric polymer to the surface of the carbon black also results in an improvement in tangent delta under test conditions predictive of a lower tire rolling resistance. Furthermore, because of the immobilization of polymer at the surface of the carbon black particles, improvements in tear and abrasion resistance also occur in rubber compounds containing coupled carbon black. Among other advantages, this combination of physical property improvements is significant due to the fact that silica's major advantage over carbon black has traditionally been in the area of improved rolling resistance and its major disadvantage (even with the use of coupling agents) has been in the area of abrasion resistance. Therefore, improving carbon black performance in both of these areas enhances the relative utility of carbon black compared to that offered by silica and minimizes the perceived advantages of silica fillers in elastomeric compositions. Additionally, these modified materials provide improved compatibility with polar elastomers, which in turn improves the processibility of these modified materials in elastomeric systems as well.

The modified carbonaceous materials of the instant application are also particularly well suited for use in waterborne systems, such as aqueous dispersions, acrylic coatings and waterborne ink formulations. For example, a modified carbon black of the present invention can be used in automotive coatings or digital ink applications. Use of these surface modified carbonaceous materials in waterborne systems offers improvements in processibility and subsequent dispersion stability. Additional performance enhancements also include jetness, undertone, and gloss. Other end use applications and corresponding advantages of the surface modified carbonaceous materials disclosed herein will be readily apparent to one of ordinary skill in the art. Moreover, the weight percent loading of modified carbonaceous materials capable of use in the above-mentioned applications will be similar to the amount of conventional carbonaceous materials presently used in these formulations and will be readily obtainable by one of ordinary skill in the art through routine experimentation.

### EXAMPLES

### Example 1

### TRIS Surface Modification of Oxidized Carbon Black

300 grains of oxidized carbon black (Raven 5000 Ultra II, obtained from Columbian Chemicals Company, Marietta, Ga. 30062 U.S.A.) was added to a 1 L round bottomed flask containing 500 mL of toluene. The flask was placed in a reflux assembly. 15 grams of N-tris(hydroxymethyl)aminomethane was dissolved in 150 mL of triethanolamine (TEA) and the resultant mixture was added to the 1 L flask containing the carbon black/toluene mixture. The resulting mixture of toluene, carbon black and N-tris(hydroxymethyl)aminomethane was refluxed at 110° C. for 12 hours, after which, the reaction was cooled to room temperature. The resulting carbon black slurry was filtered and washed several times with ethanol and then water. The washed carbon black was then dried at a temperature of 110° C. for 4 hours.

### Example 2

### TRIS Surface Modification of Oxidized Carbon Black

100 grams of oxidized carbon black (Raven 5000 Ultra II, obtained from Columbian Chemicals Company, Marietta, Ga. 30062 U.S.A) was added to a 1 L flask containing 500 mL of toluene. The flask was placed in a reflux assembly. 5 gm of N-tris(hydroxymethyl)aminomethane was dissolved in 150 mL of dimethyl ethanol amine (DMEA) and was added to the 1 L flask containing the carbon black slurry. The resultant mixture was refluxed at 110° C. for 12 hours, after which, the reaction was cooled to room temperature. The resulting carbon slurry was filtered and washed several times with ethanol and then water. The washed carbon black was then dried at 110° C. for 4 hours.

### Example 3

### TRIS Surface Modification of Oxidized Carbon Black

100 grams of oxidized carbon black (Raven 5000 Ultra II, obtained from Columbian Chemicals Company, Marietta, Ga. 30062 U.S.A) was added to a 1 L flask containing 500 mL of triethanolamine (TEA). The flask was placed in a reflux assembly. 5 g in of N-tris(hydroxymethyl)aminomethane was dissolved in 150 mL TEA and was added to the 1 L flask containing the carbon black slurry. The resultant mixture was refluxed at 110° C. for 12 hours, after which, the reaction was cooled to room temperature. The resulting carbon slurry was filtered and washed several times with ethanol and then water. The washed carbon black was then dried at 110° C. for 4 hours.

### Example 4

### Oxidation of Non-oxidized Carbon Black by Ozone Treatment

500 grams of non-oxidized carbon black powder (N234, obtained from Columbian Chemicals Company, Marietta, Ga. 30062 U.S.A) was loaded into a rotating drum. Air within the drum was enriched with gaseous ozone, to a concentration of 2 weight percent ozone, via an arc discharge in dry air using an OZAT Compact Ozone Generator Unit (made by OZONIA, Switzerland). The ozone generator was operated at 1 kilowatt of power, a pressure of 1.5 bar, and a gas flow rate of 1.4 m³/hour. The ozone enriched air was introduced into the rotating drum containing the 500 grams of non-oxidized carbon black powder and this process continued for 6 hours. After the 6 hour period was complete, the ozone generator was turned off and the drum was purged with air for 10 minutes, providing a resulting ozone oxidized carbon black composition.

### Example 5

### TRIS Surface Modification of Oxidized Carbon Black

100 grams of the oxidized carbon black prepared in Example 4 was added to a 1 L flask containing 500 mL of triethanol amine (TEA). The flask was placed in a reflux assembly. 10 gm of N-tris(hydroxymethyl)aminomethane was dissolved in a mixture of 300 mL TEA and was added to the 1 L flask containing the carbon black slurry. The resultant mixture was refluxed at 110° C. for 12 hours, after which, the reaction was cooled to room temperature. The resulting carbon slurry was filtered and washed several times with ethanol and then water. The washed carbon black was then dried at 110° C. for 4 hours.

### Example 6

### TRIS Surface Modification of Oxidized Carbon Black

10 gm of N-tris(hydroxyinethyl)aminomethane was dissolved in 200 mL of deionized water and was added to a 1 L beaker containing 100 grams of the oxidized carbon black prepared in Example 4 The resultant slurry was mixed well and heated to 190° C. for 8 hours. The resulting carbon was dispersed in 500 ml of deionized water, filtered, washed several times with ethanol and then water. The washed carbon product was then dried at 110° C. for 4 hours.

### Example 7(a)

### Waterborne Acrylic Composition Containing the Unmodified Carbon Black used to Prepare the Surface Modified Carbon Black of Example 1

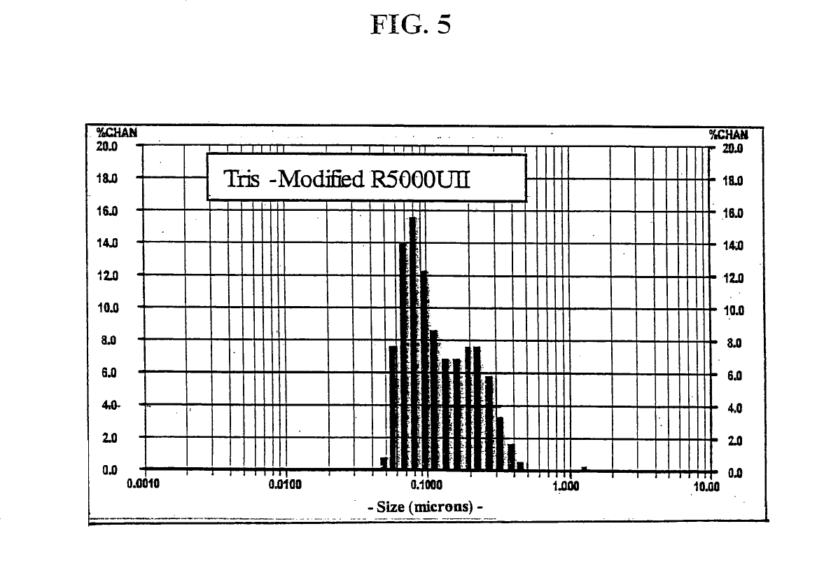

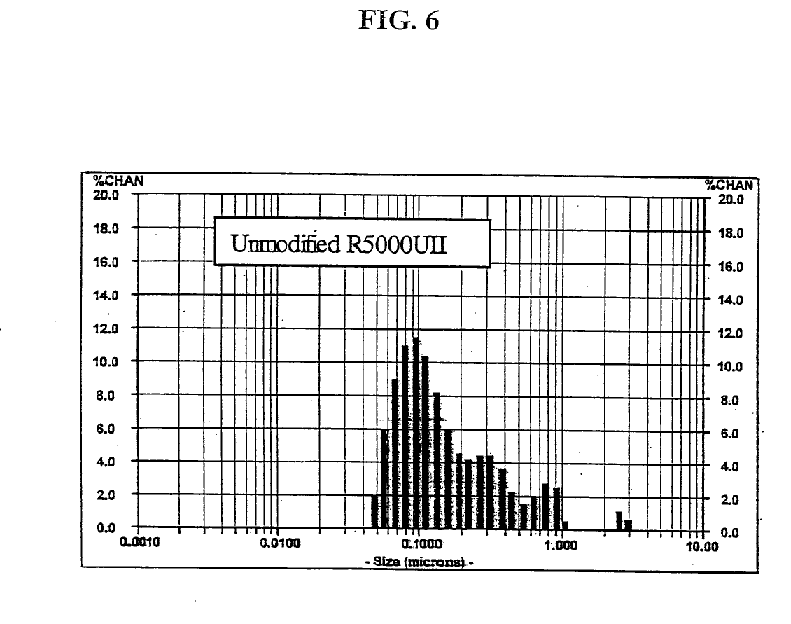

A premix was prepared by slowly adding 5 grams of the Raven 5000 Ultra II unmodified carbon black to a mixture of 35.4 grams deionized water and 14.0 grams of polyurethane resin (Borchigen SN 95, available from Bayer Corporation) using a Cowles mixer at 500 rpm for approximately 3-5 minutes. The resulting premix was transferred into a stainless steel media mill containing 460 grams of 3/32″ diameter stainless steel balls. 1.0 grams of a defoamer (Byk 021, available from Byk Chemie, Wesel, Germany), 11.2 grams of propylene glycol and 33.4 grams of acrylic latex (Neocryl A-5090, available from Neoresins, Inc., Waalwijk, The Netherlands) were also introduced into the media mill. The media mill was then placed on a paint shaker for approximately 2 hours. The resulting dispersion was then tested for particle/aggregate size and distribution, as illustrated in FIG. 6. The Hunter L, a, and b color values were also recorded on a draw down made from the dispersion, as illustrated in Table 1. Example 7(b): Waterborne Acrylic Composition Containing the TRIS Modified Carbon Black of Example 1 A premix was prepared by slowly adding 5 grams of the TRIS modified carbon black of Example 1 to a mixture of 35.4 grams deionized water and 14.0 grams of polyurethane resin (Borchigen SN 95, available from Bayer Corporation) using a cowles mixer at 500 rpm for approximately 3-5 minutes. The resulting premix was transferred into a stainless steel media mill containing 460 grams of 3/32″ diameter stainless steel balls. 1.0 grams of a defoamer (Byk 021, available from Byk Chemie, Wesel, Germany) 11.2 grams of propylene glycol and 33.4 grams of acrylic latex (Neocryl A-5090, available from Neoresins, Inc., Waalwijk, The Netherlands) were also introduced into the media mill. The media mill was then placed on a paint shaker for approximately 2 hours. The resulting dispersion was then tested for particle/aggregate size and distribution, as illustrated in FIG. 5. The Hunter L, a, and b color values were also recorded on a draw down made from the dispersion, as illustrated in Table 1.

In comparing the resulting products of Examples 7(a) and 7(b), a comparison of FIG. 6 in view of FIG. 5 indicates that the unmodified Raven 5000 Ultra II carbon black provides a waterborne acrylic composition having a larger aggregate size of dispersed carbon black as well as a broader aggregate size distribution, thus indicating that the dispersibility of the unmodified Raven 5000 Ultra II is less than that of the TRIS modified Raven 5000 Ultra II. Alternatively stated, a comparison of FIG. 5 in view of FIG. 6 indicates that the TRIS modified Raven 5000 Ultra II carbon black provides a waterborne acrylic composition having a smaller aggregate size of dispersed carbon black as well as a narrower aggregate size distribution, thus indicating the enhanced dispersibility of the TRIS modified Raven 5000 Ultra II.

Moreover, with specific reference to Table 1, it can also be seen that the unmodified RAVEN 5000 Ultra II used to prepare the acrylic composition of Example 7(a), provides a dispersion having a relatively higher Hunter L and b value, compared to the dispersion of Example 7(b). These comparative results indicate that the unmodified Raven 5000 Ultra II provides an acrylic dispersion having less black and blue color properties compared to those provided by the TRIS modified Raven 5000 Ultra II of Example 1. In other words, it can be seen that the TRIS modified RAVEN 5000 Ultra II used to prepare the acrylic composition of Example 7(b), provides a lower Hunter L and b value, thus indicating a dispersion having blacker and bluer properties respectively.

TABLE 1

| Sample # | L | a | b |
|---|---|---|---|
| Example 7(a) | 4.193 | −0.065 | −0.706 |
| Examp1e 7(b) | 4.145 | −0.030 | −0.716 |

### Example 8

### Polyurethane Coating Composition Containing TRIS Modified Carbon Black of Example 1

A polyurethane coating composition containing the TRIS modified carbon black prepared in Example 1 would be made by the following procedures.

First, a urethane-acrylic premix would be prepared by introducing 700 parts by weight of a hard aliphatic urethane emulsion and 300 parts by weight of a hard modified acrylic emulsion into a half gallon stainless steel pail and then premix on Cowles mixer at approximately 700-1000 rpm for approximately 3-5 minutes. Then while continuing to premix at approximately 700 rpm, 60 parts by weight of 2,2,4-trimethyl-1,3-pentanediol mono-2-methylpropanoate (Texanol, available from Eastman Chemical Company, Kingsport, Tenn.) and 60 parts by weight of deionized water would be added and the premix would be mixed until homogenous.

A carbon black pigment dispersion would then be prepared by adding 0.7 parts of a solution of polyether modified polysiloxane (Byk-346 available from Byk Chemie, Wesel, Germany) and 1.5 parts of potassium fluorinated alkyl carboxylate (Fluorad FC-129, available from Minnesota Mining and Manufacturing Company, St. Paul, Minn.) to 30 parts of deionized water in a beaker, while mixing on a Cowles mixer for 1-2 minutes at approximately 500-700 rpm. To the resulting mixture, 5 parts of the TRIS modified carbon black prepared in Example 1 would slowly be added at approximately 700-1000 rpm, until all the carbon black powder is wetted in. Lastly, 0.5 parts of a Byk-021 defoamer would also be added to the mixture. At this point, the resulting mixture containing the wetted carbon black powder would be transferred to a stainless steel cylinder containing 380 grams of 3/32″ stainless steel shot and then milled on a shaker for approximately 1 hour or until the resulting dispersion would provide a smooth and uniform draw down on a Leneta card using a #38 wire wound rod.

Finally, the pigmented urethane-acrylic coating composition would be prepared by slowly adding the black pigment dispersion into the urethane-acrylic premix at approximately 700-1000 rpm on a Cowles mixer for approximately 2-3 minutes. Then the coating compositions would be mixed for an additional 5 minutes at 5000 rpm to provide a finished urethane coating composition.

### Example 9

### Tire/Rubber Composition or Application Containing TRIS Modified Carbon Black of Example 1

A rubber composition comprising the TRIS modified carbon black composition prepared in Example 5 would be prepared according to the following procedures.

First, using a Banbury mixer set at a temperature of 70° C., a 77 rotor rpm, a ram pressure of 60 psi, and a 70% fill factor, a masterbatch would be prepared using the following mixing procedure:

| First Pass | |
|---|---|
| 0 seconds | Add 80 parts styrene butadiene rubber (SBR) and 20 parts |
| | polybutadiene rubber (BR) to mixer. |
| 30 seconds | Add 1-2 parts silane coupling agent, 3 parts zinc oxide, |
| | 2 parts stearic acid, and 26 parts TRIS modified N234 |
| | carbon black prepared in Example 5 to rubber mixture. |
| 90 seconds | Add 10 parts Sundex 790 aromatic oil (available from Sun |
| | Refining, Philadelphia, PA) and 26 parts TRIS modified |
| | N234 carbon black prepared in Example 5; sweep. |
| 150 seconds | Sweep. |
| 210 seconds | Sweep. |
| 240 seconds | Drop mixture to roll mill; maintain temperature |
| | above 150° C. for 60 seconds. |

After preparation of the masterbatch is complete, the masterbatch would then be milled on a 2 roll mill set to a temperature of 30° C. with both rolls set to rotate at 25 rpm.

Pass masterbatch through roll mill at a nip size of 0.050″.

Band; cross-blend six times; pass end to end 3 times.

Band at 0.050″ for 30 seconds; sheet off; lay flat, allow to cool for 1 hour.

The remaining curing agents would then be added to the masterbatch during a second pass in the Banbury mixer set at a temperature of 25° C., a 65 rotor rpm, a ram pressure of 60 psi, and a 68% fill factor, using the following procedure:

| Second Pass: | |
|---|---|
| 0 seconds | Add ½ of the masterbatch, 1.8 parts N-tert-butyl-2- |
| | benzothiazolesulfide, 0.5 parts diphenyl guanidine, and 1.9 |
| | parts sulfur; add remaining ½ masterbatch. |
| 30 seconds | Sweep |
| 120 seconds | Drop at a maximum temperature of 220° F. |

After the addition of the remaining masterbatch components is complete, the masterbatch would then be milled on a 2 roll mill set to a temperature of 30° C. with both rolls set to rotate at 25 rpm.

Pass material once through mill at a nip width of 0.050″.

Band; cross-blend six times; pass end to end 3 times.

Band at a nip width of 0.065″ for 30 seconds; sheet; lay flat to sample.

Throughout this application, where various publications are referenced, the entire disclosures of these publications are hereby incorporated by reference into this application for all purposes.

While this invention has been described in connection with preferred aspects and specific examples, it is not intended to limit the scope of the invention to the particular aspects set forth, but on the contrary, it is intended to cover such alternatives, modifications, and equivalents as may be included within the spirit and scope of the invention as defined by the appended claims. For example, there are numerous variations and combinations of components and or conditions, e.g., the carbonaceous compound, solvent, amine, reaction conditions and the like that can be used to optimize the results obtained from the described aspects. To this end, one skilled in the art will appreciate that in practicing the present invention, only reasonable and routine experimentation will be required to optimize such conditions.

What is claimed is:

1. A surface modified carbonaceous material comprising a carbonaceous material having a plurality of amide functionalities of the general formula:
—(CO)—NH—R—CR¹R²R³,
surface bonded thereto, wherein R is a single bond or a straight chain C₁-C₁₂ alkyl, and wherein R¹, R², and R³ are independently selected from C₁-C₁₂ hydroxy alkyl, C₁-C₁₂ hydroxy alkenyl, C₁-C₁₂ hydroxy alkynyl, hydrogen, hydroxyl, and C₁-C₁₂ alkyl.

2. The material of claim 1, wherein the carbonaceous material is carbon black, graphite, finely divided carbon, activated charcoal, fullerenic carbon, or nanocarbon

3. The material of claim 1, wherein the carbonaceous material is carbon black.

4. The material of claim 1, wherein R is a single bond and wherein at least one of R¹, R², and R³ comprises a hydroxyl susbtituent.

5. The material of claim 1, wherein R is a single bond and wherein at least two of R¹, R², and R³ comprise a hydroxyl substituent.

6. The material of claim 1, wherein R is a single bond and wherein each of R¹, R², and R³ individually represents a hydroxy methyl substituent.

7. The material of claim 1, wherein the carbonaceous material has a surface area of at least approximately 200 m²/g.

8. The material of claim 1, wherein the surface atomic concentration of oxygen is at least approximately 8.0% relative to the total surface atomic concentration of the surface treated carbonaceous material.

9. The material of claim 1, wherein the surface atomic concentration of nitrogen is at least approximately 0.1% relative to the total surface atomic concentration of the surface treated carbonaceous material.

10. A process for the manufacture of a surface modified carbonaceous material comprising a carbonaceous material having a plurality of amide functionalities of the general formula —(CO)—NH—R—CR¹R²R³, surface bonded thereto, the process comprising the steps of:
a) providing a carbonaceous material comprising a plurality of carboxylic acid functional groups surface bonded thereto; and
b) reacting the carbonaceous material with an amine of the general formula H₂N—R—CR¹R²R³, wherein R is a single bond or straight chain C₁-C₁₂ alkyl, and wherein R¹, R², and R³ are independently selected from C₁-C₁₂ hydroxy alkyl, C₁-C₁₂ hydroxy alkenyl, C₁-C₁₂ hydroxy alkynyl, hydrogen, hydroxyl, and C₁-C₁₂ alkyl;
wherein the reaction of the carbonaceous material with the amine proceeds under conditions effective to provide a surface modified carbonaceous material comprising a carbonaceous material having a plurality of amide functionalities of the general formula —(CO)—NH—R—CR¹R²R³, surface bonded thereto, wherein R is a single bond or straight chain C₁-C₁₂ alkyl, and wherein R¹, R², and R³ are independently selected from C₁-C₁₂ hydroxy alkyl, C₁-C₁₂ hydroxy alkenyl, C₁-C₁₂ hydroxy alkynyl, hydrogen, hydroxyl, and C₁-C₁₂ alkyl.

11. The process of claim 10, wherein the carbonaceous material is carbon black, graphite, finely divided carbon, activated charcoal, fullerenic carbon or nanocarbon.

12. The process of claim 10, wherein the carbonaceous material is carbon black.

13. The process of claim 10, wherein the carbonaceous material of a) is an oxidized carbonaceous material.

14. The process of claim 10, wherein R is a single bond and wherein at least one of R¹, R², and R³ comprises a hydroxyl susbtituent.

15. The process of claim 10, wherein R is a single bond and wherein at least two of R¹, R², and R³ comprise a hydroxyl substituent.

16. The process of claim 10, wherein the amine is Tris(hydroxymethyl)amino methane.

17. The process of claim 10, wherein the reaction takes place in the presence of a suitable solvent.

18. The process of claim 17, wherein the suitable solvent comprises dimethylethanolamine.

19. The process of claim 17, wherein the suitable solvent comprises toluene, xylene or a mixture thereof.

20. The process of claim 10, wherein the reaction takes place in the absence of a solvent.

21. The process of claim 10, wherein greater than approximately 70% of the plurality of carboxylic acid functional groups of a) react with the amine of b) to provide the plurality of amide groups.

22. The process of claim 10, wherein the surface modified carbonaceous material comprising a plurality of amide functionalities has a surface atomic concentration of oxygen that is at least approximately 20.0% greater than the surface atomic concentration of the carbonaceous material of a).

23. An aqueous composition, comprising the surface modified carbonaceous material of claim 1 and water.

24. The composition of claim 23, wherein the composition is an aqueous dispersion and wherein the surface modified carbonaceous material is a surface modified carbon black.

25. An elastomeric composition, comprising the carbonaceous material of claim 1 and an elastomer.

26. The composition of claim 25, wherein the elastomer is rubber.

27. The composition of claim 25, wherein the elastomeric composition is suitable for use in manufacturing tires.

28. The product produced by the process of claim 10.
